(19) United States
(12) Patent Application Publication
(10) Pub. No.: US 2008/0177341 A1
(43) Pub. Date: Jul. 24, 2008

(54) Automated external defibrillator (AED) system with multiple patient wireless monitoring capability for use in mass casualty incidents

(76) Inventor: Kyle R. Bowers, Boxborough, MA (US)

Correspondence Address: Mark J. Pandiscio;Pandiscio & Pandiscio, P.C., 470 Totten Pond Road, Waltham, MA, 02451-1914, US

(21) Appl. No.: 11/978,261

(22) Filed: Oct. 29, 2007

Related U.S. Application Data
(60) Provisional application No. 60/854,915, filed on Oct. 27, 2006.

(51) Int. Cl. A61B 5/04; A61N 1/00

(52) U.S. Cl. 607/5; 607/142; 600/509

(57) ABSTRACT

An Automated External Defibrillator (AED) with wireless patient monitoring capability. The AED is used in conjunction with a monitoring chest strap that transmits the patient's ECG and other parameters over a wireless network to the AED. The AED is capable of monitoring several patients simultaneously for use in mass casualty incidents. The AED notifies and indicates to the operator when a patient requires defibrillation therapy. The device is ready to shock once the defibrillation electrodes are applied.

### REFERENCE TO PENDING PRIOR PATENT APPLICATION

This patent application claims benefit of pending prior U.S. Provisional Patent Application Ser. No. 60/854,915, filed Oct. 27, 2006 by Kyle R. Bowers for AUTOMATED EXTERNAL DEFIBRILLATOR (AED) SYSTEM WITH MONITORING CAPABILITY (Attorney's Docket No. ACCESS-9 PROV), which patent application is hereby incorporated herein by reference.

### FIELD OF THE INVENTION

This invention relates to Automated External Defibrillators (AEDs) in general, and more particularly to Automated External Defibrillators (AEDs) with patient monitoring capability.

### BACKGROUND OF THE INVENTION

Approximately 350,000 deaths occur each year in the United States alone due to Sudden Cardiac Arrest (SCA). Worldwide deaths due to Sudden Cardiac Arrest (SCA) are believed to be at least twice that of the United States. Many of these deaths can be prevented if effective defibrillation is administered within 3-5 minutes of the onset of SCA.

Sudden Cardiac Arrest (SCA) is the onset of an abnormal heart rhythm, lack of pulse and absence of breath, leading to a loss of consciousness. If a pulse is not restored within a few minutes, death occurs. Most often, SCA is due to Ventricular Fibrillation (VF), which is a chaotic heart rhythm that causes an uncoordinated quivering of the heart muscle. The lack of coordinated heart muscle contractions results in a lack of blood flow to the brain and other organs. Unless this chaotic heart rhythm is quickly terminated, thereby allowing the heart to restore its own normal rhythm, death ensues.

Rapid defibrillation is the only known means to restore a normal heart rhythm and prevent death after SCA due to Ventricular Fibrillation (VF). For each minute that passes after the onset of SCA, mortality typically increases by 10%. At 7-10 minutes, the survival rate is generally below 10%. However, if a patient is effectively defibrillated within 1-2 minutes of the onset of SCA, survival rates can be as high as 90% or more. Therefore, the only known way to increase the chance of survival for an SCA victim is through early defibrillation.

Automated External Defibrillators (AEDs) offer the prospect of such early defibrillation, but they must be (i) portable so they can be easily carried to an SCA victim, (ii) easy-to-use so that they can be properly utilized when SCA occurs, and (iii) easily maintained.

### SUMMARY OF THE INVENTION

The present invention provides a new AED with, among other things, patient monitoring capability.

In accordance with one preferred form of the present invention, the new AED contains a set of electrodes that are applied directly to the patient from the defibrillator. The electrodes contain an electrically conductive hydrogel that adheres the patient's skin. The defibrillator uses the electrodes to sense ElectroCardioGram (ECG) signals from the patient so as to determine the condition of the patient's heart and hence identify a shockable or non-shockable condition. The defibrillator also uses the electrodes to sense the patient's transthoracic impedance so as to determine the appropriate shock parameters. If a shockable condition is indicated, the defibrillator applies a pulsed voltage potential at the electrodes, which causes a flow of electrical current through the patient's chest.

In accordance with one preferred form of the present invention, the AED contains a shock delivery circuit, which is used to deliver an appropriate biphasic shock to the patient.

In accordance with one preferred form of the present invention, the shock delivery circuit contains a battery, high voltage capacitors, a circuit to charge the capacitors from the battery, and a circuit to deliver a biphasic waveform from the capacitors to the patient.

In accordance with one preferred form of the present invention, the AED contains an ECG and impedance analysis circuit to determine if the patient requires therapy and to measure and analyze the patient's transthoracic impedance, so that the therapeutic waveform is delivered to the patient in a controlled and accurate manner.

In accordance with one preferred form of the present invention, the AED contains a user interface to facilitate interaction with the user and to guide the user through a sequence of rescue events.

In accordance with one preferred form of the present invention, the AED user interface provides buttons which may be used to control the device.

In accordance with one preferred form of the present invention, the AED user interface contains a high-resolution Liquid Crystal Display (LCD), voice playback circuitry, an audio amplifier and a speaker, all of which may be used to guide the rescuer through a resuscitation effort.

In accordance with one preferred form of the present invention, the AED contains a controller circuit which operates the device.

In accordance with one preferred form of the present invention, the controller circuit contains one or more microprocessors, microcontrollers, memory, and other circuitry to enable AED operation.

In accordance with one preferred form of the present invention, the AED contains memory to store information about the patient, such as the patient's ECG data. The memory may be internal, external or removable.

In accordance with one preferred form of the present invention, the AED utilizes a second set of electrodes which are used to monitor the patient's ECG.

In accordance with one preferred form of the present invention, the AED may be configured to monitor patient parameters other than ECG, e.g., patient pulse, patient temperature, patient blood pressure, patient blood oxygen level, etc.

In accordance with one preferred form of the present invention, the system includes a patient monitor which incorporates a second set of electrodes for monitoring the patient's ECG and/or other sensors for monitoring parameters other than ECG, e.g., patient pulse, patient temperature, patient blood pressure, patient blood oxygen level, etc. In one preferred form of the present invention, the patient monitor comprises a patient monitoring cable which is hard-wired to the AED. In another preferred form of the invention, the patient monitor comprises a wireless patient monitor which is wirelessly connected to the AED.

In accordance with one preferred form of the present invention, the AED has a monitoring mode of operation where the user can observe the patient's ECG or other parameter while the patient is being transported.

In accordance with one preferred form of the present invention, the AED is capable of communicating with a computer. The communications link is used to transfer data to and from the AED.

In one preferred form of the present invention, the AED communicates with the computer using a hard-wire connection.

In one preferred form of the present invention, the AED communicates with the computer via a wireless connection.

In one preferred form of the present invention, there is provided an automated external defibrillator (AED) for applying a therapeutic bi-phasic shock pulse to a patient, said automated external defibrillator (AED) comprising:

a battery;

a plurality of capacitors for storing charge from the battery;

a pair of defibrillation electrodes for positioning on the exterior of the chest of the patient;

patient monitor apparatus comprising a pair of monitoring sensors for positioning on the exterior of the patient; and

control circuitry interposed between (i) the battery and the plurality of capacitors, and (ii) the plurality of capacitors and the pair of defibrillation electrodes, the control circuitry being configured to:
(1) monitor patient parameters using the patient monitor apparatus;
(2) using the pair of monitoring sensors, determine if the patient is in a shockable condition;
(3) selectively charge the plurality of capacitors from the battery;
(4) measure the thoracic impedance of the patient by applying a non-therapeutic, assessment pre-pulse to the patient and then terminating the same, wherein the non-therapeutic, assessment pre-pulse comprises a brief discharge of selected ones of the plurality of capacitors, the non-therapeutic, assessment pre-pulse having (i) a sufficiently low voltage and a sufficiently low current, applied for a sufficiently short time duration, as to deliver a safe, non-therapeutic, assessment current to the patient, and (ii) a duration long enough to obtain an accurate reading of the patient's thoracic impedance but short enough to avoid substantially depleting the capacitors;
(5) calculate the thoracic impedance of the patient from the non-therapeutic, assessment pre-pulse applied to the patient;
(6) determine the level of energy to be applied to the patient in the therapeutic bi-phasic shock pulse;
(7) determine the number of capacitors to be discharged into the patient, and the duration of the discharge, based upon the calculated thoracic impedance of the patient and the level of energy to be applied to the patient in the therapeutic bi-phasic shock pulse, so as to provide a therapeutic bi-phasic shock pulse to the patient; and
(8) provide a therapeutic bi-phasic shock pulse to the patient, by discharging the determined number of capacitors, for the determined duration, into the pair of defibrillation electrodes positioned on the exterior of the chest of the patient.

In another preferred form of the present invention, there is provided patient monitor apparatus comprising:

a strap;

a pair of monitoring sensors secured to the strap for positioning on the exterior of the patient for monitoring patient parameters when the strap is secured to the patient; and

data transmission apparatus for transmitting patient parameters to an AED.

In still another form of the present invention, there is provided a method for treating a patient, wherein the method comprises:

providing an automated external defibrillator (AED) for applying a therapeutic bi-phasic shock pulse to a patient, said automated external defibrillator (AED) comprising:
a battery;
a plurality of capacitors for storing charge from the battery;
a pair of defibrillation electrodes for positioning on the exterior of the chest of the patient;
patient monitor apparatus comprising a pair of monitoring sensors for positioning on the exterior of the patient; and
control circuitry interposed between (i) the battery and the plurality of capacitors, and (ii) the plurality of capacitors and the pair of defibrillation electrodes, the control circuitry being configured to:
   (1) monitor patient parameters using the patient monitor apparatus;
   (2) using the pair of monitoring sensors, determine if the patient is in a shockable condition;
   (3) selectively charge the plurality of capacitors from the battery;
   (4) measure the thoracic impedance of the patient by applying a non-therapeutic, assessment pre-pulse to the patient and then terminating the same, wherein the non-therapeutic, assessment pre-pulse comprises a brief discharge of selected ones of the plurality of capacitors, the non-therapeutic, assessment pre-pulse having (i) a sufficiently low voltage and a sufficiently low current, applied for a sufficiently short time duration, as to deliver a safe, non-therapeutic, assessment current to the patient, and (ii) a duration long enough to obtain an accurate reading of the patient's thoracic impedance but short enough to avoid substantially depleting the capacitors;
   (5) calculate the thoracic impedance of the patient from the non-therapeutic, assessment pre-pulse applied to the patient;
   (6) determine the level of energy to be applied to the patient in the therapeutic bi-phasic shock pulse;
   (7) determine the number of capacitors to be discharged into the patient, and the duration of the discharge, based upon the calculated thoracic impedance of the patient and the level of energy to be applied to the patient in the therapeutic bi-phasic shock pulse, so as to provide a therapeutic bi-phasic shock pulse to the patient; and
   (8) provide a therapeutic bi-phasic shock pulse to the patient, by discharging the determined number of capacitors, for the determined duration, into the pair of defibrillation electrodes positioned on the exterior of the chest of the patient;

applying the patient monitor apparatus to the patient and connecting the patient monitor apparatus to the control circuitry,

monitoring patient parameters using the patient monitor apparatus;

if a shockable condition is detected in the patient, defibrillating the patient using the AED.

### BRIEF DESCRIPTION OF THE DRAWINGS

These and other objects and features of the present invention will be more fully disclosed or rendered obvious by the following detailed description of the preferred embodiments of the present invention, which are to be considered together with the accompanying drawings wherein like numbers refer to like elements and further wherein:

FIG. 1 is a schematic view of the new AED and its electrodes attached to the patient;

FIG. 2 is a block diagram showing a high-level system diagram of the new AED;

FIG. 3 is a block diagram showing more detailed system diagram of the new AED system;

FIG. 4 is a schematic view showing the front of the new AED;

FIG. 5 is a block diagram showing the processor/co-processor architecture of the new AED;

FIG. 6 is a diagram of the AED shock waveform;

FIG. 7 is a schematic view of the AED battery;

FIG. 8 is a schematic view of the AED battery having a cutout for a USB communications connection;

FIG. 9 is a schematic view of the AED with wireless USB adapter installed;

FIG. 10 is an example of the AED's status indication system conditions;

FIG. 11 is an exemplary flow diagram showing a preferred method for charging the AED capacitors;

FIG. 12 is a schematic view showing a wireless patient ECG monitor strap that contains two electrodes;

FIG. 13 is a schematic view showing another wireless patient ECG monitor strap system that contains two electrodes;

FIG. 14 is a schematic view showing a wireless patient ECG monitor strap that contains two flying electrode leads;

FIG. 15 is a schematic view showing a wireless patient ECG monitor strap that contains three flying electrode leads;

FIG. 16 is a schematic view showing a wireless patient ECG monitor strap that contains two electrodes within the strap and a third flying electrode lead; and

FIG. 17 is a side view showing the new AED's multi-media card and USB connections.

### DETAILED DESCRIPTION OF THE PREFERRED EMBODIMENTS

In accordance with the present invention, the new AED is provided with monitoring capability. This permits the user to monitor the patient's condition after resuscitation or in the event the patient is breathing and does not require a shock. Among other things, the new AED may be used to monitor the patient while the patient is being transported to the hospital.

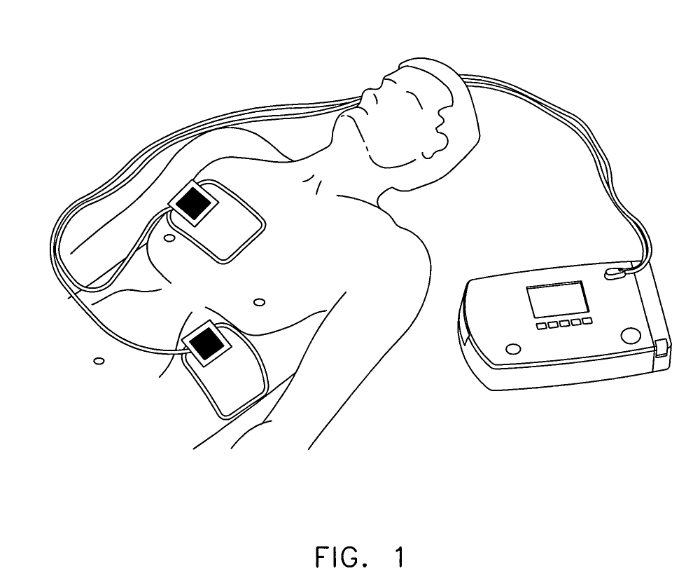

A typical connection of the AED to the patient is shown in FIG. 1.

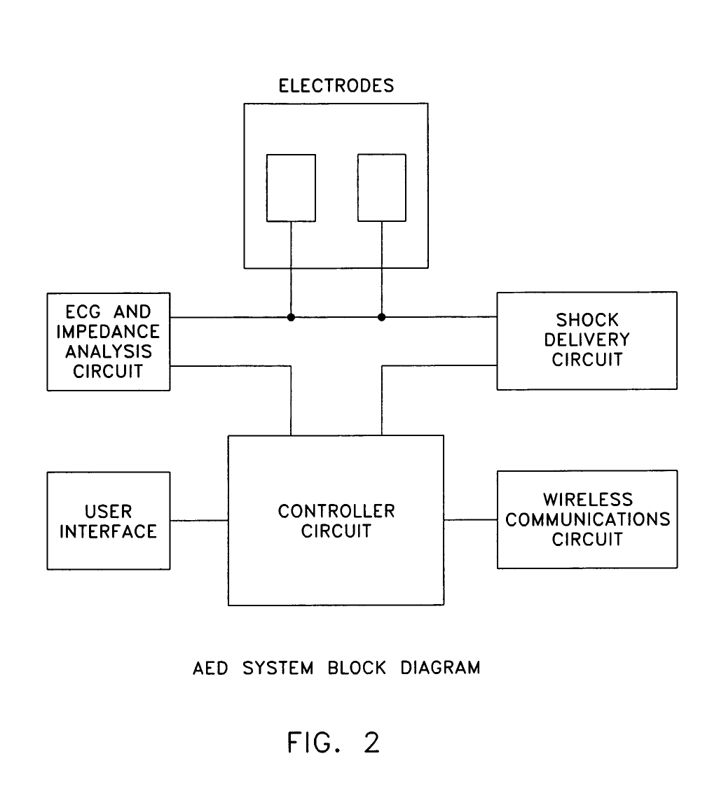

The present invention comprises an Automated External Defibrillator (AED) as shown in the high level system block diagram in FIG. 2.

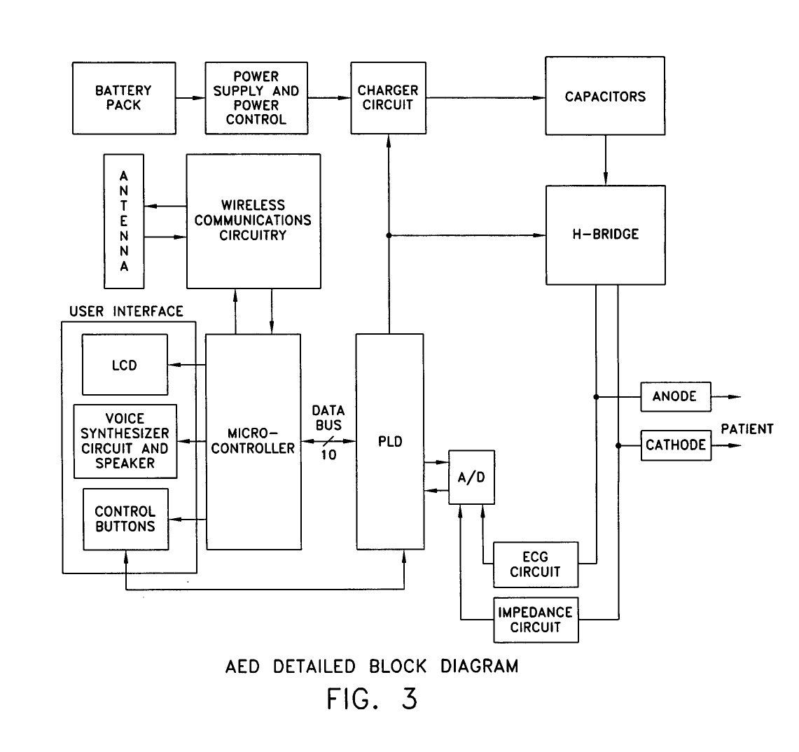

A more detailed block diagram of the new AED is shown in FIG. 3.

The AED also contains the necessary components for defibrillation including, but not limited to, a battery pack, capacitor charger circuit, high-voltage capacitors and an H-bridge circuit (see FIG. 3).

The defibrillator also contains several other components such as, but not limited to, a real-time clock, analog-to-digital converters, digital-to-analog converters, operational amplifiers, audio amplifiers, random access memory, dynamic random access memory, flash memory, electrically erasable read only memory and other memories as well (including internal, external and removable).

The defibrillator also contains a high-resolution LCD screen, voice synthesizer circuit and speaker for instructing the rescuer during device use.

In one preferred embodiment of the present invention, the defibrillator LCD screen may be a color TFT display or similar technology, capable of displaying text, graphics, high-resolution images and video. In accordance with the present invention, the pictures and video may be used to show details or demonstrations for instructional purposes. One example is the device may be used to show a video clip to the user on how to perform CPR.

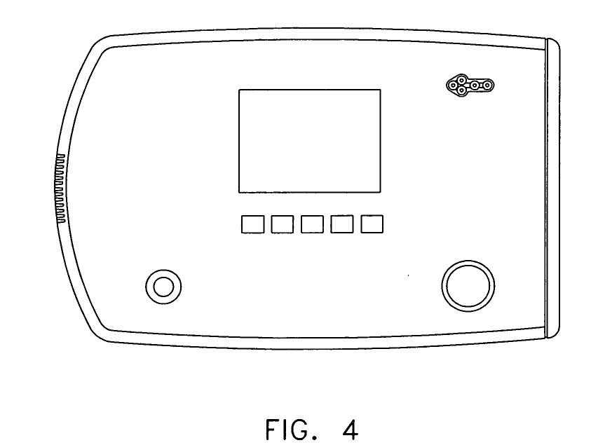

The defibrillator also includes several buttons for user control. These buttons may comprise, but are not limited to, a power button, a shock button and several special purpose buttons located below the display. The special purpose buttons act as “soft keys”, i.e., keys which are defined by displaying their function in text on the display, immediately above the button, as shown in FIG. 4. The soft keys are, therefore, fully programmable and have unlimited possibilities for functionality.

The defibrillator contains a status indication system to alert the user of the readiness of the device. The status indication system contains a visual indicator, a buzzer, a voice playback circuit and a speaker.

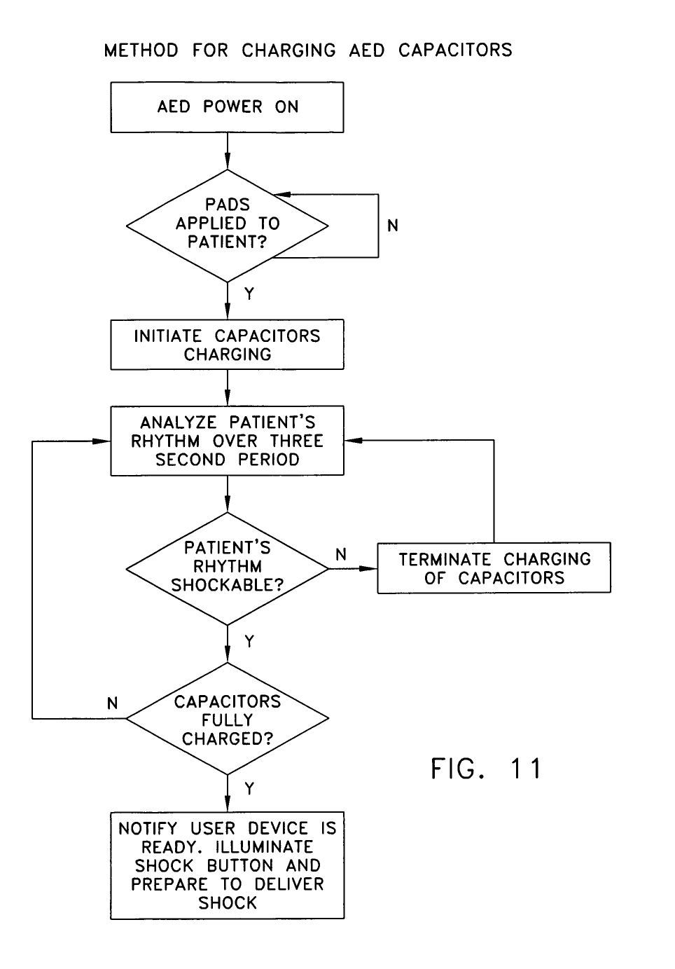

In one preferred embodiment of the present invention, the indicator has three colors; green, yellow and red to notify the user of the readiness of the device. In accordance with the present invention, the indicator colors denote the following conditions: (1) the device is ready to use, (2) the device requires maintenance, but can be used, and (3) the device has failed and should not be used. These indicators may blink, and/or be accompanied by one or more audio tones to alert the rescuer. These indicators may also be accompanied by voice prompts, visual prompts or audio tones. A table showing exemplary conditions for the status indicators is shown in FIG. 10.

The defibrillator contains controllers for operating the defibrillator. These controllers may include microprocessors, microcontrollers, digital signal processors, field programmable gate arrays, programmable logic devices, and other digital or analog circuitry.

In accordance with the present invention, the defibrillator contains a watch-dog timer circuit. The watch-dog timer circuit monitors operation of the main controller circuit of the defibrillator during all modes of operation.

In one preferred embodiment of the present invention, the watch-dog timer circuit contains its own controller.

In another preferred embodiment of the present invention, the status indication system also contains its own controller.

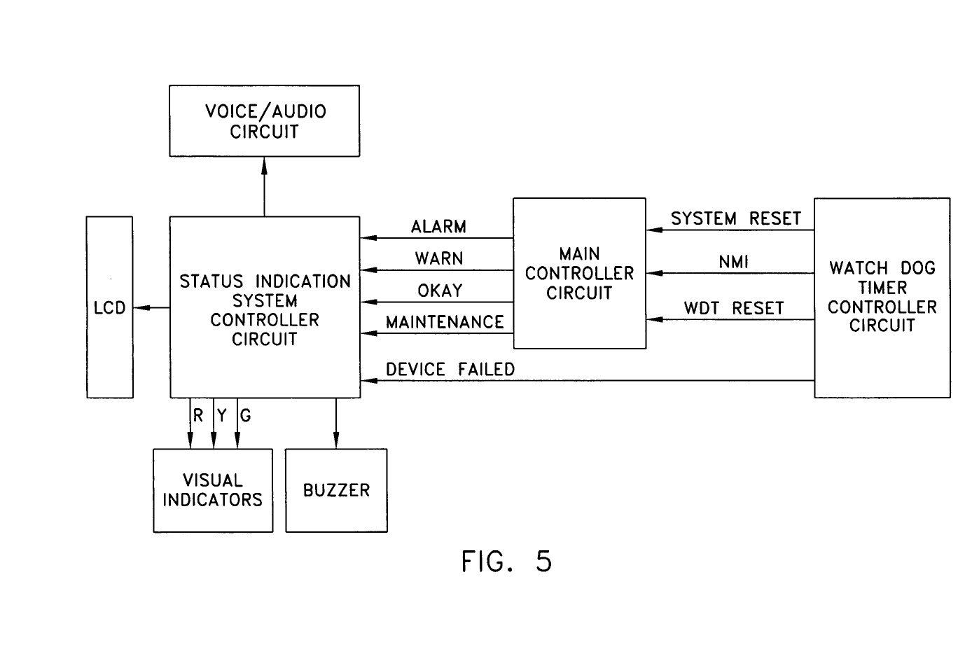

In another preferred embodiment of the present invention, the main controller, watch-dog timer circuit and status indication system are arranged in a processor/co-processor architecture as shown in FIG. 5.

In accordance with the present invention, the watch-dog timer controller first attempts to restart the main processor if it stops operating or fails. If the main controller does not resume operation, the watch-dog timer circuit then resets the entire system. If the main controller still does not resume operation after the system reset, the watch-dog timer controller sends a signal to the status indication system controller to indicate that the device has failed and should not be used.

In accordance with the present invention, the main controller may send a signal to the status indication system controller to indicate its internal status, the results of self-test, the status of the device's peripherals and other relevant signals.

In accordance with the present invention, the watch-dog timer controller or status indication system controllers are configured so that if either fail, the status indication system indicates that the device is unusable, ensuring the entire system is failsafe.

The AED is very simple to operate. Once the electrode pads are removed from their pouch and installed on the patient, the device automatically analyzes the patient's heart rhythm. If the AED determines that the patient's heart rhythm is shockable (i.e., requiring defibrillation therapy), the AED charges the capacitors and notifies the user that a shock is advised and to stand clear from the patient.

In one preferred embodiment of the present invention, the AED begins to charge the capacitors once the electrodes have been applied to the patient. The AED considers the electrodes to be applied to the patient once the AED measures the patient's impedance and determines that the patient's impedance is within a specified range. One example of an acceptable patient impedance range is 20 to 200 ohms.

The AED continues to charge the capacitors and analyze the patient's heart rhythm for a period of time. In one preferred embodiment of the present invention, the AED charges the capacitors and analyzes the heart rhythm for a period of three seconds. If the AED determines that the patient's heart rhythm is not shockable, charging of the capacitors is terminated. If, however, the rhythm is determined to be shockable, the AED continues to charge the capacitors until the target voltage is reached. An example of this method is shown in the flow diagram in FIG. 11. It should be noted that after the first three second period, the AED considers combinations of analyzed periods (i.e., 2 out of 3 shockable periods) to determine if the device shall continue to charge the capacitors.

Once the capacitors are charged and the device is ready, the shock button is illuminated and the rescuer simply presses the shock button to deliver a shock. The AED senses the patient's impedance, determines the appropriate shock parameters, and then delivers the therapeutic shock to the patient.

FIG. 1 is a pictorial diagram of the AED applied to the patient.

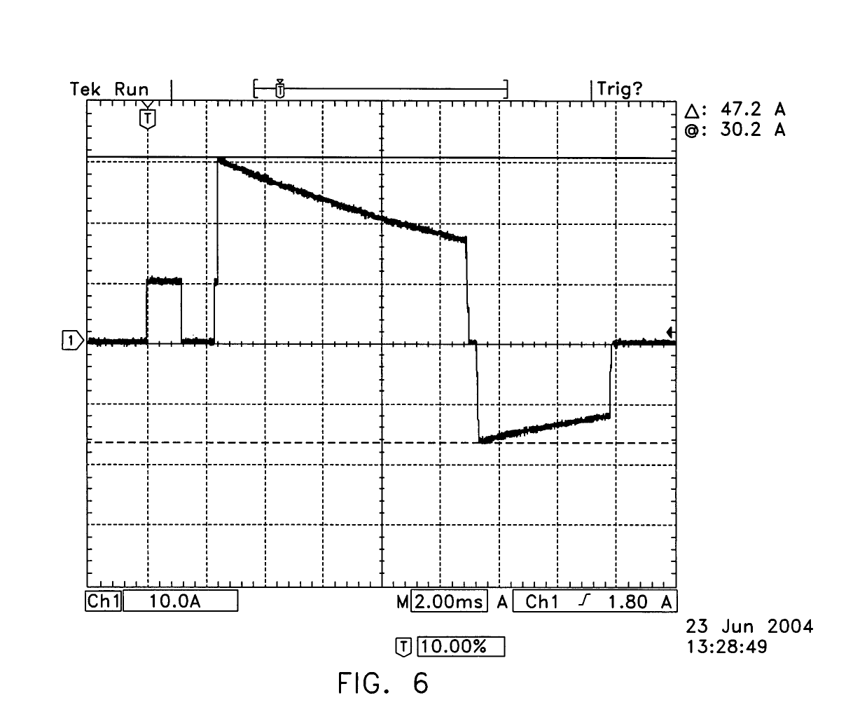

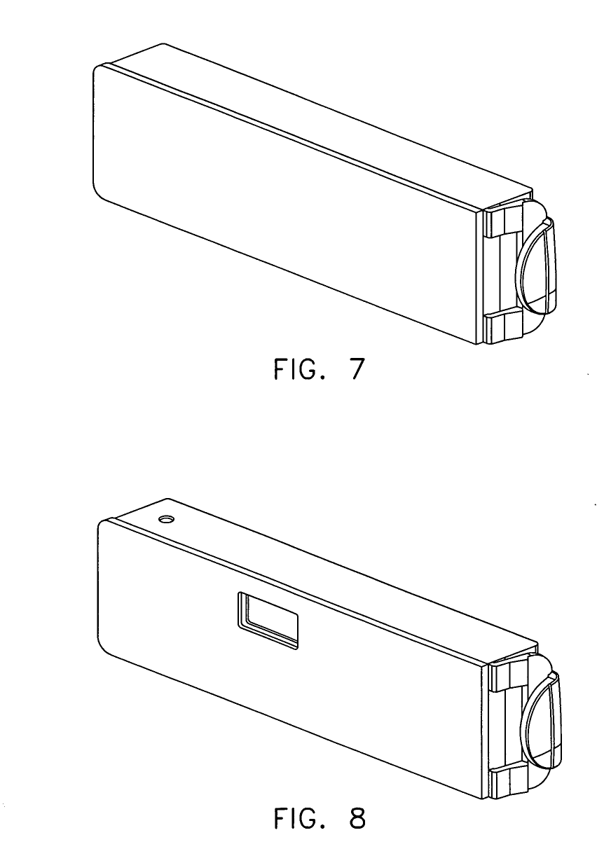

FIG. 6 is a diagram showing the AED biphasic shock waveform. As shown in FIG. 6, the AED uses a discrete sensing pulse to determine the patient's impedance using large-signal current before the therapeutic waveform is generated.

In one embodiment of the present invention, the AED uses multiple capacitors which are specifically configured to provide a desired impedance-compensated biphasic shock waveform. The controller uses the impedance measurement acquired during the pre-pulse, and a look-up table in memory, to determine the capacitor configuration to be used. The capacitors are then arranged in series/parallel combinations, using switches, so as to provide safe and optimal shock parameters, i.e., controlling the shock voltage and limiting peak currents in low-impedance patients. The AED achieves this by providing lower shock voltages for low-impedance patient's (i.e., using parallel capacitor configuration), therefore limiting the peak current. In high-impedance patients, the AED provides higher shock voltages (i.e., using series capacitor configuration) and lengthens the duration of the biphasic waveform.

In another preferred embodiment of the present invention, the AED uses the small-signal impedance measurement to determine the patient's impedance prior to charging the capacitors. The target voltage is then calculated according to the measured patient impedance. In accordance with the present invention, the AED measures the patient impedance and configures the capacitors as described in the embodiment above, however, it uses a simplified configuration look-up table. Those skilled in the art can appreciate that the AEDs circuitry is also minimized by this approach, by providing less configurations of the capacitors, leading to a lower cost defibrillator. In accordance with the present invention, the AED also accurately delivers the intended amount of energy.

In yet another preferred embodiment of the present invention, the controller, by using both the small-signal impedance measurement to set the target voltage and the large-signal pre-pulse measurement to set the capacitor configuration, and by adjusting the duration of the phases of the waveform, can achieve an optimal charge ratio. As is well known in the art, a biphasic waveform comprising a phase 2-to-phase 1 charge ratio of approximately 0.38 produces optimal efficacy.

In accordance with the present invention, the AED notifies the user that a shock has been delivered to the patient. After the shock has been delivered, the AED prompts the user to begin CPR for a period of time.

In one preferred embodiment of the present invention, the CPR period is programmable and can be configured by a medical director before the AED is placed in service.

In accordance with the present invention, the CPR period is programmable from 30 seconds to 3 minutes, but could be easily modified to include other periods as well.

After the AED has completed the CPR interval, the device prompts the user to stand clear and begins to analyze the patient's heart rhythm. If the patient requires an additional shock, the shock/CPR sequence is repeated.

If the patient does not require a shock, the AED will repeat the CPR interval, then prompt the user to stand clear and re-analyze the patient's heart rhythm.

In accordance with the present invention, the AED delivers the first shock at 200 joules (J).

In one preferred embodiment of the present invention, the AED can be configured by the medical director to escalate the energy on the second shock.

In one preferred embodiment of the present invention, the AED escalates to 360 joules (J) after the first shock.

In another preferred embodiment of the present invention, the AED is configurable to escalate to 250 J, 300 J, 330 J or 360 J after the first shock.

In accordance with the present invention, the AED contains internal memory which records data during the resuscitation event. This data includes, but is not limited to, the patient's ECG, transthoracic impedance, the results of the analyzed heart rhythm, user prompts, how long the device was used, how many and when shocks were delivered, the results of self-test and many other parameters.

In accordance with the present invention, the system includes a patient monitor which incorporates a second set of electrodes for monitoring the patient's ECG and/or other sensors for monitoring parameters other than ECG, e.g., patient pulse, patient temperature, patient blood pressure, patient blood oxygen level, etc. In one preferred form of the present invention, the patient monitor comprises a patient monitoring cable which is hard-wired to the AED. In another preferred form of the invention, the patient monitor comprises a wireless patient monitor which is wirelessly connected to the AED.

In one preferred form of the present invention, the AED is capable of being used with a patient monitor which contains ECG electrodes. The patient monitor is initially positioned on the patient and the ECG is monitored using the AED. If the AED determines that the patient is experiencing SCA and is shockable, the AED alerts the rescuer and the patient monitoring cable is removed and replaced by the AED's defibrillation electrodes. The AED then operates in the normal manner, i.e., it monitors heart rhythm and defibrillates when appropriate. This embodiment of the invention has the significant advantage that when a patient is initially encountered and it is unclear whether the patient is experiencing SCA and is shockable, the patient monitor (with inexpensive ECG electrodes) can be used for patient assessment before the rescuer needs to open a sealed package of the relatively expensive defibrillator electrodes.

In accordance with the present invention, the AED enters into monitor mode when it detects the activation of the patient monitor, i.e., a patient monitoring cable and/or a wireless patient monitor.

In one preferred embodiment of the present invention, the patient monitoring cable comprises a 3-Lead ECG, for selection of either Lead I or Lead II.

In another preferred embodiment of the present invention, the patient monitoring cable comprises 3 Leads, but it used to monitor Lead II only and comprises a right leg drive signal.

In yet another preferred embodiment of the present invention, the patient monitoring cable comprises a 2-Lead ECG and monitors Lead II only.

In yet another preferred embodiment of the present invention, the patient monitoring cable comprises a 4-Lead ECG, for selection of either Lead I or Lead II and comprises a right leg drive signal.

In accordance with the present invention, the AED, when used in monitor mode, may additionally record heart rate, heart rate alarms, when alarms are reset or suspended, user prompts and other events.

In accordance with the present invention, the AED, when in monitor mode, uses the soft keys to control the heart rate alarms, lead selection, ECG gain and set of menus to change the operating mode.

In one preferred embodiment of the present invention, the AED, while in monitor mode, analyzes the patient's ECG signal. If the AED determines that the patient's heart rhythm is shockable, it prompts the user to replace the patient monitoring cable with the defibrillation electrodes. Once the defibrillation electrodes are detected, the device switches to AED mode and prepares to deliver a shock.

In one preferred embodiment of the present invention, the AED, while in monitor mode, begins to charge the capacitors once it determines that the patient's heart rhythm is shockable.

In another preferred embodiment of the present invention, the AED, while in monitor mode, continues to charge the capacitors once it has detected a shockable rhythm. The AED continues to charge the capacitors until fully charged. The AED continues to charge the capacitors even if the patient monitoring cable has been removed and the defibrillation electrodes are in the process of being applied to the patient.

In accordance with the present invention, once the defibrillation electrodes are correctly applied to the patient, the AED analyzes the heart rhythm for period of time, and then illuminates the shock button and prompts the user to press the shock button if the heart rhythm is determined to be shockable. In one preferred embodiment of the present invention, the period of time that the AED analyzes the patient's heart rhythm before prompting the user to press the shock button is three seconds.

In one preferred embodiment of the present invention, the AED comprises wireless monitoring capability. More particularly, the AED contains wireless circuitry and antenna that communicate with a wireless patient monitor. Thus, the AED need not be within cable length of the patient, as with the patient monitoring cable construction.

The patient monitoring cable and/or the wireless patient monitor primarily transmits ECG data to the AED.

In one preferred embodiment of the present invention, the patient monitoring cable and/or the wireless patient monitor is capable of sending additional data such as the patient's transthoracic impedance signal.

In another preferred embodiment of the present invention, the communications between the AED and the patient monitoring cable and/or the wireless patient monitor is two way. The AED may send requests for data to the patient monitoring cable and/or the wireless patient monitor, such as, but not limited to, requests for status signals, requests for self-tests results, requests for resend of data, and commands, such as to place the patient monitoring cable and/or the wireless patient monitor in sleep mode.

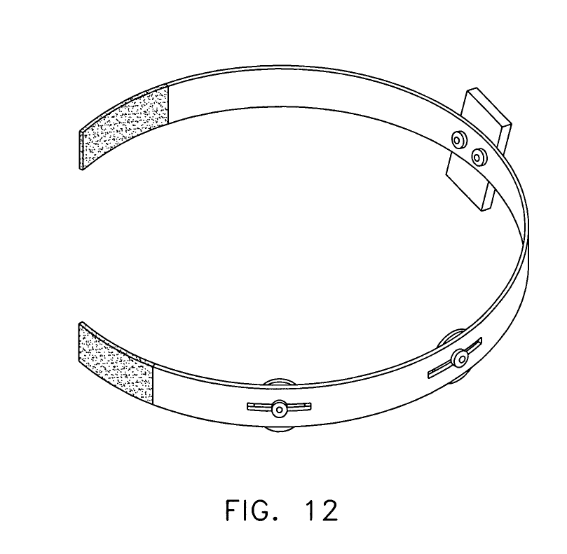

In another preferred embodiment of the present invention, the patient monitoring cable and/or the wireless patient monitor is embedded into a strap, which goes around the patient's chest (FIG. 12). The transmitter/receiver is also attached to the strap and the ECG leads run through the center of the strap. The electrodes are mounted to rails, which make the position adjustable to correctly locate the electrode according to the size of the patient's chest.

In accordance with the present invention, the ECG electrodes are snap-on and replaceable, but other types of ECG electrodes may also be used such as, but not limited to, non-replaceable types and metal electrodes.

In one preferred embodiment of the present invention, the strap is made of a stretchable fabric and is secured to the patient's chest by Velcro ends. However, other types of ends could also be used, such as buckle, snap buckle and belt type ends with the use of a strap adjustment (not shown).

In accordance with the present invention, the patient monitoring cable and/or the wireless patient monitor is attached to the patient so that its transmitter/receiver is positioned on the side of the patient's torso, but could be located on any part of the strap and could also be moveable.

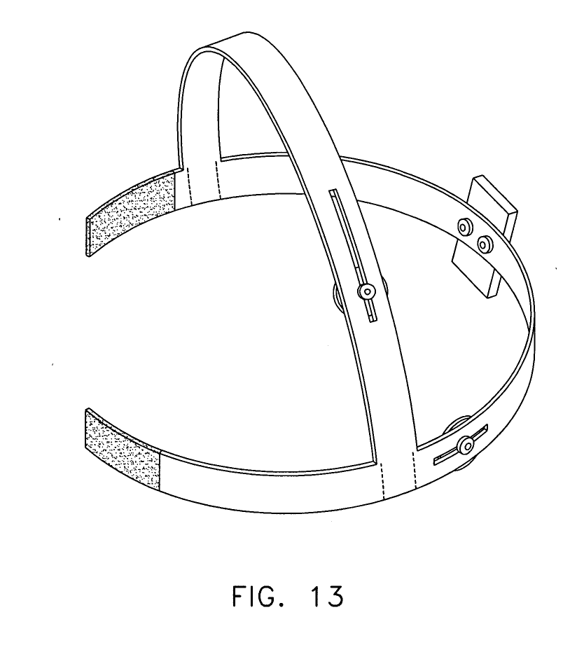

In another preferred embodiment of the present invention, the patient monitoring cable and/or the wireless patient monitor has a second strap which wraps over the patient shoulder and contains the second electrode (FIG. 13). The second strap may be permanently fixed (as shown) or attachable using the methods discussed previously.

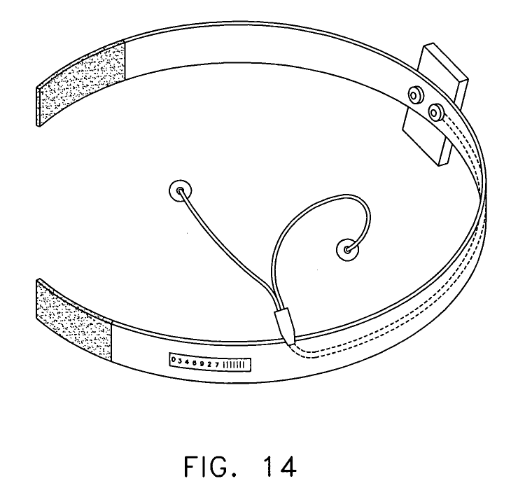

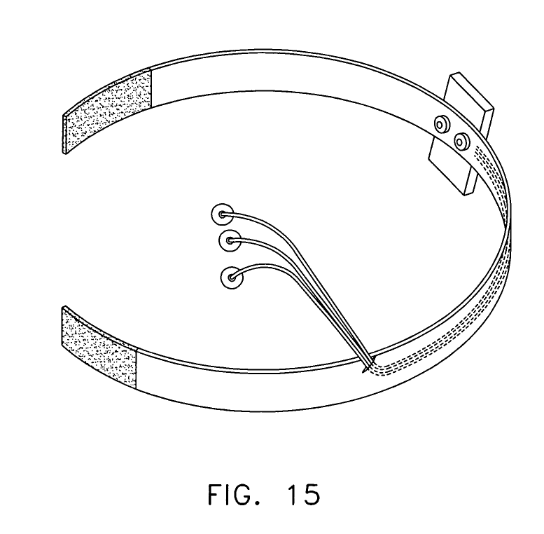

In yet another preferred embodiment of the present invention, the patient monitoring cable and/or the wireless patient monitor includes a strap has an opening where the “flying” electrode leads emerge and may be positioned on the patient's chest. In accordance with the present invention, the monitor strap may be two lead (FIG. 14) or three lead (FIG. 15).

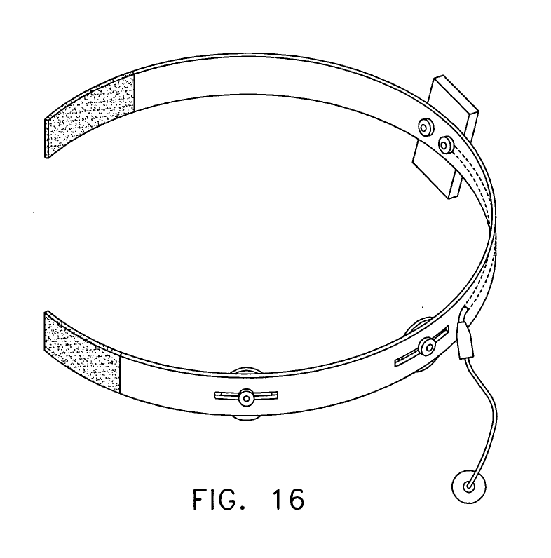

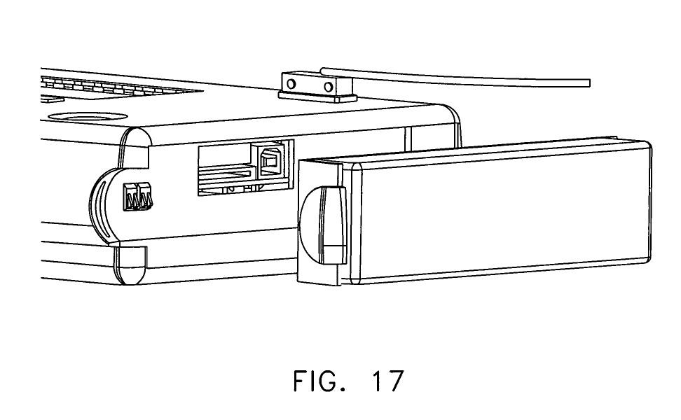

In yet another preferred embodiment of the present invention, the patient monitor cable and/or the wireless patient monitor includes a strap has a combination of embedded electrodes and flying leads, as shown in the conceptual example of FIG. 16.

In a preferred embodiment of the present invention, the patient monitor cable and/or the wireless patient monitor includes a strap has an ID tag (FIG. 14) that may contain numbers, letters, bar-codes, magnetic strips and/or other identification components or markings.

In accordance with the present invention, the AED, while used as a patient monitor, will identify the patient being monitored on the display using the ID tag information, so the user may identify the patient currently being monitored.

In another preferred embodiment of the present invention, the AED may be used to monitor one or more patients at a time. This construction is extremely valuable in situations where a single rescuer may need to urgently triage and/or monitor a number of patients, e.g., in the case of a battlefield situation involving multiple wounded, a terrorist incident involving multiple victims, a transportation accident involving multiple injured (e.g., an airplane crash, a train accident, a multi-car accident, etc.), etc. In this case, the AED prompts the user when an additional patient monitor cable and/or wireless patient monitor is detected. The user may then switch to display the ECG of the detected patient. The AED prompts the user to enter information into the AED about the patient such as, but not limited to, the patient's name, age, sex, height, weight, race, known medical conditions, sensitivity to medications, etc. As previously described, the patient monitor cable and/or wireless patient monitor contains an ID number tag, so the user can identify the patient being monitored.

In accordance with the present invention, the AED displays only one ECG at a time, but continues to record the patient's ECG and other monitored data as described above, to the AED's memory. The AED uses the patient's ID (e.g., the patient's name) as an icon, with a soft key to allow the user to easily switch back and forth between patients.

It should be appreciated that when viewed in one way, the present invention comprises an AED with the ability to monitor the ECG and/or other parameters, of a plurality of patients at the same time and to provide life-saving defibrillation when necessary.

Viewed another way, the present invention comprises a multi-patient emergency response system with both patient monitoring and defibrillation capability.

In accordance with the present invention, the AED displays only one ECG at a time, but continues to monitor all active patient's and prompts the user if a patient needs attention, such as when a shockable rhythm is detected. The AED also prompts the user if it is unable to communicate with a previously-active patient monitor cable and/or a previously-active wireless patient monitor. Such examples might occur where the cable is damaged or the wireless device is out of range, the device is turned off, the device is removed from the patient, or any other reason that the central monitor is unable to communicate with previously-active device.

In accordance with the present invention, when the patient monitor cable and/or the wireless patient monitor is removed from the patient, the AED prompts the user to remove the patient from the monitoring screen and terminates data recording.

In accordance with the present invention, the AED also has a manual mode of operation for professionally trained users that want full control of the defibrillator.

In accordance with the present invention, the AED, when in manual mode, uses the soft keys to control the charging of the capacitors, synchronization to the patient's R-wave, and energy delivery selection.

In accordance with the present invention, the AED, while used in a resuscitation event or while used to monitor a patient, records the patient's ECG signal and many other events and data. In a preferred embodiment of the present invention, the AED contains a universal serial bus (USB) interface that allows connection to a desktop PC, laptop PC and/or other type of computer device that supports USB.

In one preferred embodiment of the present invention, the AED is powered by the PC, etc. through the USB connection. In one preferred form of the invention, the AED's battery must be removed to access the USB connector, as shown in FIG. 17. The AED cannot be operated when connected to the PC, ensuring no safety hazard while used in this mode.

In accordance with the present invention, the PC, etc. runs a proprietary computer program, which allows the recorded data to be uploaded for event review. The computer program translates the data and displays the information (e.g., the patient's ECG) graphically.

In accordance with the present invention, the AED firmware can also be upgraded by the PC, etc. over the USB interface. In one preferred embodiment of the present invention, specific memory components may be upgraded or reprogrammed by the PC, etc. over the USB interface. An example of one type of memory component is the AED's native language prompts that are audible and visible.

In accordance with the present invention, the AED also contains configurable operational parameters. In one preferred embodiment of the present invention, the AED's operational parameters may be configured by a medical director prior to the device being placed into service. In another preferred embodiment of the present invention, the medical director creates a signature including, but not limited to, the name of the medical director, the director's affiliation, when the device was configured, and contact information of the director. This signature is stored permanently into the AED's configuration log, which is stored internally in the device. In yet another preferred embodiment of the present invention, the signature is stored permanently on the PC's configuration log along with other information including, but not limited to, the AED's serial number, the device's native language and other operational parameters, the name of the organization the device was placed in service with and when the device was placed into service, how long the device has been in service, the number of shocks delivered by the device, the number and results of self-tests and any faults recorded by the device. This allows for historical tracking of the AED by the factory or by specially trained personnel.

In another preferred embodiment of the present invention, the AED uses a removable memory device so as to store the patient and device data as described above. In one preferred embodiment of the present invention, the AED uses a multi-media card (MMC) to record the data. The MMC card is installed into the device when it placed into service.

In accordance with the present invention, the MMC card may be removed from the AED and placed in a PC card reader, which enables the PC to uploaded the data from the MMC to the PC. The proprietary computer program described above allows the user to review the stored records.

In another preferred embodiment of the present invention, the user may install the MMC card into the AED after the device has been placed into service and used. A special menu allows the user to transfer the records stored in the internal flash memory to the MMC card. In accordance with the present invention, the AED allows the user to erase the records stored in the internal flash memory of the AED, once the records have successfully been transferred to the MMC card. In accordance with the present invention, the computer program allows the user to erase the contents of the MMC.

In another preferred embodiment of the present invention, the user may install the MMC card after the AED has been placed into service. In accordance with the present invention, the AED automatically transfers the stored data to the MMC and erases the internally stored records, once the data transfer is successfully completed. In one preferred embodiment of the present invention, the AED transfers the data to the MMC and erases the internal memory after it has successfully completed the device's daily self-test. As those skilled in the art can appreciate, the transfer and/or erasing of large blocks of data in memory types such flash memories may take many seconds or minutes depending on the size of the memory. In accordance with the present invention, the AED performs this data transfer when the AED is least likely to be used. If the AED is used during any part of the transfer/erase cycle, the device terminates this transfer/erase mode and resumes on the next daily self-test.

In accordance with the present invention, the AED may be used in a training mode.

In one preferred embodiment of the present invention, the AED is connected to a PC via the USB communications interface to enable training mode. In a preferred embodiment of the present invention, the AED is powered by the PC through the USB connection. The AED's battery must be removed to access the USB connector as shown in FIG. 17. The device can not be operated as an AED when connected to the PC, ensuring no safety hazard while used in this mode.

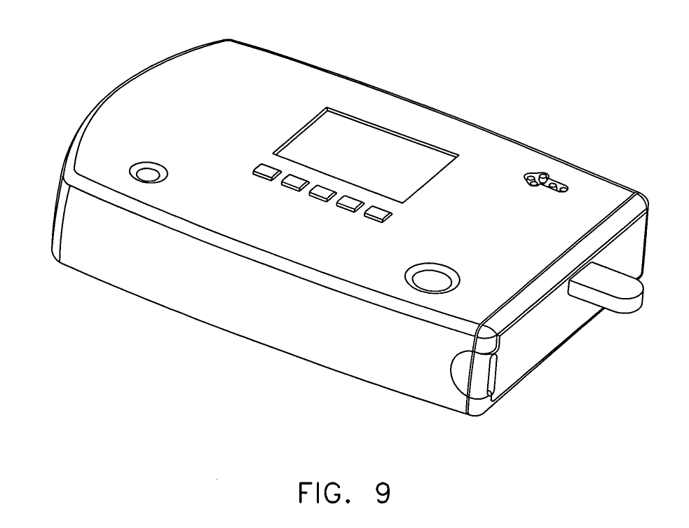

In another preferred embodiment of the present invention, the AED has a special training battery as shown in FIG. 8. The training battery consists of rechargeable and/or consumable battery cells. The rechargeable version contains a plug for connection to a low-voltage wall-transformer type power supply. The training battery capacity is limited and the device cannot be operated as an AED when connected to the PC, ensuring no safety hazard while used in this mode.

In yet another preferred embodiment of the present invention, the AED includes a wireless communications circuit and is capable of communicating with the PC over a wireless network, such as Bluetooth, Wi-Fi or other types of wireless networks. The device may only be used in training mode while the special training battery is installed as described above.

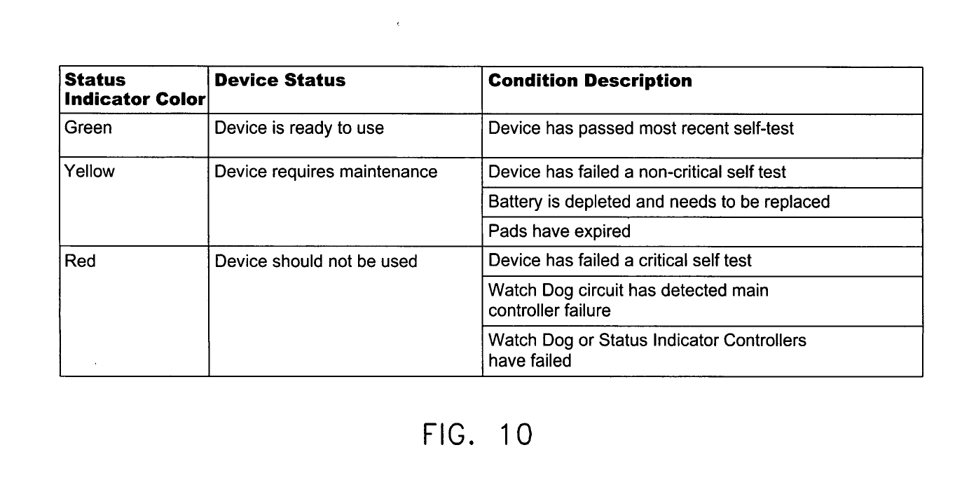

In yet another preferred embodiment of the present invention, the AED uses a wireless USB adapter to communicate to the PC over a wireless network as described above. The device may only be used in training mode while the special training battery is installed as described above. The training battery has a cut-away which allows the adapter to be plugged in, as shown in FIG. 9.

In accordance with the present invention, the PC runs a proprietary computer program that allows the AED to run in training mode.

In a preferred embodiment of the present invention, the computer program allows the training instructor to control one or more AEDs over the wireless network. This allows the instructor to teach an entire class at once. In accordance with the present invention, the AED, while in training mode, runs scripts used to teach the user how to operate the device. The instructor may cause the AEDs to start, stop, reset or switch scripts during a training session.

### MODIFICATIONS

While the present invention has been described in terms of certain exemplary preferred embodiments, it will be readily understood and appreciated by those skilled in the art that it is not so limited, and that many additions, deletions and modifications may be made to the preferred embodiments discussed herein without departing from the scope of the invention.

What is claimed is:

1. An automated external defibrillator (AED) for applying a therapeutic bi-phasic shock pulse to a patient, said automated external defibrillator (AED) comprising:
a battery;
a plurality of capacitors for storing charge from the battery;
a pair of defibrillation electrodes for positioning on the exterior of the chest of the patient;
patient monitor apparatus comprising a pair of monitoring sensors for positioning on the exterior of the patient; and
control circuitry interposed between (i) the battery and the plurality of capacitors, and (ii) the plurality of capacitors and the pair of defibrillation electrodes, the control circuitry being configured to:
(1) monitor patient parameters using the patient monitor apparatus;
(2) using the pair of monitoring sensors, determine if the patient is in a shockable condition;
(3) selectively charge the plurality of capacitors from the battery;
(4) measure the thoracic impedance of the patient by applying a non-therapeutic, assessment pre-pulse to the patient and then terminating the same, wherein the non-therapeutic, assessment pre-pulse comprises a brief discharge of selected ones of the plurality of capacitors, the non-therapeutic, assessment pre-pulse having (i) a sufficiently low voltage and a sufficiently low current, applied for a sufficiently short time duration, as to deliver a safe, non-therapeutic, assessment current to the patient, and (ii) a duration long enough to obtain an accurate reading of the patient's thoracic impedance but short enough to avoid substantially depleting the capacitors;
(5) calculate the thoracic impedance of the patient from the non-therapeutic, assessment pre-pulse applied to the patient;
(6) determine the level of energy to be applied to the patient in the therapeutic bi-phasic shock pulse;
(7) determine the number of capacitors to be discharged into the patient, and the duration of the discharge, based upon the calculated thoracic impedance of the patient and the level of energy to be applied to the patient in the therapeutic bi-phasic shock pulse, so as to provide a therapeutic bi-phasic shock pulse to the patient; and
(8) provide a therapeutic bi-phasic shock pulse to the patient, by discharging the determined number of capacitors, for the determined duration, into the pair of defibrillation electrodes positioned on the exterior of the chest of the patient.

2. An automatic external defibrillator (AED) according to claim 1 wherein the control circuitry is adapted to begin charging the capacitors when the defibrillation electrodes have been applied to the patient.

3. An automatic external defibrillator (AED) according to claim 2 wherein the control circuitry is adapted to stop charging the capacitors after a pre-calculated period of time, if the patient's analyzed rhythm is determined to be non-shockable.

4. An automatic external defibrillator (AED) according to claim 1 wherein the control circuitry is adapted to begin monitoring patient parameters when it detects activation of the patient monitor.

5. An automatic external defibrillator (AED) according to claim 1 wherein the control circuitry begins charging the capacitors when a shockable rhythm is detected.

6. An automatic external defibrillator (AED) according to claim 1 wherein the patient monitor apparatus is hard-wired to the control circuitry.

7. An automatic external defibrillator (AED) according to claim 1 wherein the patient monitor apparatus is wirelessly connected to the control circuitry.

8. An automatic external defibrillator (AED) according to claim 1 wherein the patient parameters monitored by the patient monitor apparatus comprise at least one selected from the group consisting of: ECG, pulse, temperature, blood pressure, and blood oxygen level.

9. An automatic external defibrillator (AED) according to claim 1 wherein the patient monitor apparatus comprises at least two pairs of monitoring sensors for positioning on the exterior of at least two patients.

10. An automatic external defibrillator (AED) according to claim 9 wherein the control circuitry is adapted to monitor at least two patients simultaneously.

11. An automatic external defibrillator (AED) according to claim 1 wherein the control circuitry is adapted to send commands to the patient monitor apparatus.

12. An automatic external defibrillator (AED) according to claim 1 wherein the control circuitry alerts a user when a patient parameter satisfies a selected criteria.

13. An automatic external defibrillator (AED) according to claim 1 wherein the control circuitry alerts a user when the patient monitor apparatus is no longer reliably reporting patient parameters to the control circuitry.

14. An automatic external defibrillator (AED) according to claim 1 wherein the battery is configured for limited-functionality for safe use in a training setting.

15. Patient monitor apparatus comprising:
a strap;
a pair of monitoring sensors secured to the strap for positioning on the exterior of the patient for monitoring patient parameters when the strap is secured to the patient; and
data transmission apparatus for transmitting patient parameters to an AED.

16. Patient monitor apparatus according to claim 15 wherein the data transmission apparatus is hard-wired to the AED.

17. Patient monitor apparatus according to claim 15 wherein the data transmission apparatus is wirelessly connected to the AED.

18. Patient monitor apparatus according to claim 15 wherein the patient parameters monitored comprise at least one selected from the group consisting of: ECG, pulse, temperature, blood pressure, and blood oxygen level.

19. A method for treating a patient, wherein the method comprises:
providing an automated external defibrillator (AED) for applying a therapeutic bi-phasic shock pulse to a patient, said automated external defibrillator (AED) comprising:
a battery;
a plurality of capacitors for storing charge from the battery;
a pair of defibrillation electrodes for positioning on the exterior of the chest of the patient;
patient monitor apparatus comprising a pair of monitoring sensors for positioning on the exterior of the patient; and
control circuitry interposed between (i) the battery and the plurality of capacitors, and (ii) the plurality of capacitors and the pair of defibrillation electrodes, the control circuitry being configured to:
(1) monitor patient parameters using the patient monitor apparatus;
(2) using the pair of monitoring sensors, determine if the patient is in a shockable condition;
(3) selectively charge the plurality of capacitors from the battery;
(4) measure the thoracic impedance of the patient by applying a non-therapeutic, assessment pre-pulse to the patient and then terminating the same, wherein the non-therapeutic, assessment pre-pulse comprises a brief discharge of selected ones of the plurality of capacitors, the non-therapeutic, assessment pre-pulse having (i) a sufficiently low voltage and a sufficiently low current, applied for a sufficiently short time duration, as to deliver a safe, non-therapeutic, assessment current to the patient, and (ii) a duration long enough to obtain an accurate reading of the patient's thoracic impedance but short enough to avoid substantially depleting the capacitors;
(5) calculate the thoracic impedance of the patient from the non-therapeutic, assessment pre-pulse applied to the patient;
(6) determine the level of energy to be applied to the patient in the therapeutic bi-phasic shock pulse;
(7) determine the number of capacitors to be discharged into the patient, and the duration of the discharge, based upon the calculated thoracic impedance of the patient and the level of energy to be applied to the patient in the therapeutic bi-phasic shock pulse, so as to provide a therapeutic bi-phasic shock pulse to the patient; and
(8) provide a therapeutic bi-phasic shock pulse to the patient, by discharging the determined number of capacitors, for the determined duration, into the pair of defibrillation electrodes positioned on the exterior of the chest of the patient;
applying the patient monitor apparatus to the patient and connecting the patient monitor apparatus to the control circuitry;
monitoring patient parameters using the patient monitor apparatus;
if a shockable condition is detected in the patient, defibrillating the patient using the AED.

20. A method according to claim 19 wherein the patient monitor apparatus is hard-wired to the AED.

21. A method according to claim 19 wherein the patient monitor apparatus is wirelessly connected to the AED.

22. A method according to claim 19 wherein the patient parameters monitored comprise at least one selected from the group consisting of: ECG, pulse, temperature, blood pressure, and blood oxygen level.

23. A method according to claim 19 wherein the patient monitor apparatus comprises at least two pairs of monitoring sensors for positioning on the exterior of at least two patients.

24. A method according to claim 23 wherein the control circuitry is adapted to monitor at least two patients simultaneously.
